(19) United States
(12) Patent Application Publication
(10) Pub. No.: US 2024/0138752 A1
(43) Pub. Date: May 2, 2024

(54) SEPSIS RISK ASSESSMENT AND TREATMENT

(71) Applicant: Hill-Rom Services, Inc., Batesville, IN (US)

(72) Inventors: Swathi UTUKURI, Marcellus, NY (US); Eugene URRUTIA, Apex, NC (US); Patrick NOFFKE, Hartland, WI (US); Christopher L. LONG, Chittenango, NY (US); Thomas MYERS, Syracuse, NY (US); Rebecca QUILTY-KOVAL, Baldswinville, NY (US)

(21) Appl. No.: 18/490,992

(22) Filed: Oct. 20, 2023

Related U.S. Application Data
(60) Provisional application No. 63/381,207, filed on Oct. 27, 2022.

(51) Int. Cl. A61B 5/00; G16H 10/60

(52) U.S. Cl. CPC A61B 5/412; A61B 5/4839; A61B 5/7275; A61B 5/742; G16H 10/60

(57) ABSTRACT

A patient support apparatus includes a controller, a sensor capable of detecting vital signs, and a point of care (PoC) device for assessing risk of developing sepsis and for providing local and/or remote indications to caregivers. The controller is coupled to the sensor and the point of care device, and the controller can analyze signals from the sensor and the point of care device to determine data indicative of sepsis.

### PRIORITY CLAIM

This application claims priority under 35 U.S.C. § 119(e) to U.S. Provisional Application No. 63/381,207, filed Oct. 27, 2022, which is expressly incorporated by reference herein.

### BACKGROUND

The present disclosure relates to the use of sensors and point of care devices of a patient support apparatus, such as a hospital bed, for example, to determine if a patient has sepsis. The present disclosure is also directed to a system or method for accurately determining sepsis risk in a patient, accurate fluid management for the septic patient, and communicating a therapy operation protocol to a caregiver.

Patients in healthcare facilities may have a risk of developing sepsis during their stay. Sepsis is a negative condition or response that a person's body has to an infection. Sepsis results when a person's immune system stops fighting or has an insufficient response to an infection Sepsis risk assessments of patients oftentimes take place on a sporadic basis with prolonged periods transpiring between the assessments. Furthermore, the results of risk assessments are sometimes only available at a limited number of locations in the healthcare facility such as at an electronic medical records computer or at a computer of a master nurse station. Accordingly, there is a need in the healthcare field to have more timely information regarding the risk assessments of sepsis and there is a need for the risk assessment information to be more readily available to caregivers.

Fluid administration is a fundamental sepsis therapy and usually comprises a sepsis bundle fluid challenge. However, if the fluid leaks into interstitial space instead of entering the circulatory system, it may cause more harm than good to the patient. Thus, there is a need to increase the accuracy of fluid management in sepsis patients.

Many patient supports include a frame, a deck supported by the frame, a mattress, and side rails configured to block egress of a patient from the mattress, and a controller configured to control one or more features of the bed. Monitoring the various components of the patient support apparatus may include monitoring for the position, angle, activity, or other patient attributes. A caregiver may be not be able to determine if the different components are not in compliance with a therapy operation protocol. The ability of the patient support apparatus to determine compliance automatically may be important to prevent any potential harm to patients. Thus, the characterization of the components of the patient support apparatus and patient conditions should be determined and communicated to the caregiver to provide optimal care for the patient.

### SUMMARY

The present disclosure includes one or more of the features recited in the appended claims and/or the following features which, alone or in any combination, may comprise patentable subject matter.

According to a first aspect of the present disclosure, is a patient support apparatus comprising a sensor providing a first signal indicative of a vital sign of a patient positioned on the patient support apparatus, a point of care device providing a second signal, and a controller coupled to the sensor and the point of care device, including a processor and a memory device, the memory device including instructions that, when executed, cause the controller to receive the first signal from the sensor and the second signal from the point of care device, analyze the first and the second signals to determine data indicative of sepsis, compare the data indicative of sepsis to pre-established acceptable limits, and if the patient is assessed to have a risk for sepsis or has been diagnosed with sepsis, output a command signal.

In one embodiment, the patient support apparatus further comprises a sepsis notification system that responds to the command signal from the controller to display a first visual indication that shows if the patient has risk for sepsis or has been diagnosed with sepsis. In one embodiment, the point of care device is a peripheral device or a connected device. In one embodiment, the point of care device is a cell counter, a cell quality monitor, or a cardiac monitor. In one embodiment, the point of care device is a blood test module and comprises disposable tools for testing blood cell count or blood cell quality. In one embodiment, the point of care device can acquire white blood cell count of the patient in less than about a minute.

In one embodiment, the sepsis notification system responding to the command signal from the controller displays a second visual indication that shows if the patient needs fluid administration. In one embodiment, the second visual indication further indicates an amount of fluid required for fluid administration.

In one embodiment, the controller is configured to communicate with an external nurse call station. In one embodiment, the controller is configured to communicate with an electronic medical record system to receive information from the electronic medical record system indicative of a medical history of a patient supported on the patient support apparatus.

According to second aspect of the present disclosure is a patient support apparatus comprising a sensor providing a first signal indicative of a first vital sign of a patient supported on the patient support apparatus, a point of care device providing a second signal indicative of a cardiac output of the patient, and a controller coupled to the sensor and the point of care device, and including a processor and a memory device, the memory device including instructions that, when executed, cause the controller to receive the first and second signals, calculate a sepsis risk severity and an optimal fluid challenge amount based on the signals.

In one embodiment, the controller further receives and analyzes information from an electronic medical record storing patient information for the patient. In one embodiment, the apparatus further comprises a notification system responding to the command signal from the controller to provide a first visual indication that shows that the patient has risk for sepsis or has been diagnosed with sepsis. In one embodiment, the apparatus further comprises a notification system responding to the command signal from the controller to provide a first indication that shows that the patient has risk for sepsis or has been diagnosed with sepsis, and a second indication that shows an amount of fluid needed for fluid administration.

In one embodiment, the apparatus further comprises the controller communicating with a patient infusion system to automatically adjust fluid input to the patient. In one embodiment, the notification system provides a third indication that shows a sepsis graph indicating a rate of improvement in the patient. In one embodiment, the controller is configured to communicate with an external nurse call station. In one embodiment, the controller can communicate with the sensor and the point of care device though a wireless communication.

According to a third aspect of the present disclosure is a method of predicting sepsis in a patient on a patient support apparatus comprising an inflatable mattress, the method comprising the steps of monitoring a first signal from a sensor, wherein the first signal is indicative of a vital sign of the patient, monitoring a second signal from a point of care device, processing the first signal and the second signal to determine data indicative of sepsis, comparing the data indicative of sepsis to pre-established acceptable limits to assess if the patient has sepsis or has a risk for sepsis, and outputting a command signal to indicate if the patient has sepsis or has a risk for sepsis.

In one embodiment, the method comprises processing the first signal and the second signal to determine data indicative of sepsis comprises a controller analyzing the first and the second signals to determine data indicative of sepsis and comparing the data indicative of sepsis to pre-established acceptable limits. In one embodiment, processing the first signal and the second signal further comprises the controller receiving and analyzing information from an electronic medical record storing patient information for the patient.

In one embodiment, the method further comprises a notification system responding to the command signal from the controller to provide a first visual indication that shows that the patient has sepsis or has a risk for sepsis. In one embodiment, the method further comprises the notification system responding to the command signal from the controller to provide a second visual indication that shows if the patient needs fluid administration. In one embodiment, the method further comprises the notification system responding to the command signal from the controller to provide a third visual indication that shows an amount of fluid needed for fluid administration. In one embodiment, the method further comprises the controller communicating with a patient infusion system to automatically adjust fluid input to the patient. In one embodiment, the method further comprises the notification system responding to the command signal from the controller to provide a fourth visual indication that shows a sepsis graph indicating a rate of improvement in the patient.

In one embodiment, the method further comprises the controller communicating with an external nurse call station. In one embodiment of the method, the point of care device is a peripheral device or a connected device.

### BRIEF DESCRIPTION OF THE DRAWINGS

The detailed description particularly refers to the accompanying figures in which:

FIG. 1 is a perspective view of a patient support apparatus including a sepsis monitoring unit according to the present disclosure;

FIG. 2 is a perspective view of the patient support apparatus of FIG. 1 including a plurality of devices included in the sepsis-status notification system used to display the indication;

FIG. 3 is a flowchart describing a process of determining the presence of sepsis and providing an indication of the presence of sepsis using a sepsis-status notification system;

FIG. 4 is a flowchart describing a process of determining fluid administration to a sepsis patient and providing an indication using a sepsis-status notification system; and

FIG. 5A is an enlarged view of an indicator panel coupled to the patient support apparatus displaying a visual indication that the patient is septic;

FIG. 5B is an enlarged view of the indicator panel indicating that the patient needs fluid administration;

FIG. 5C is an enlarged view of the indicator panel indicating the amount of fluid that the patient needs; and

FIG. 5D is an enlarged view of the indicator panel showing a sepsis graph indicating a rate of improvement in the patient and indicating fluid responsiveness of the patient.

### DETAILED DESCRIPTION

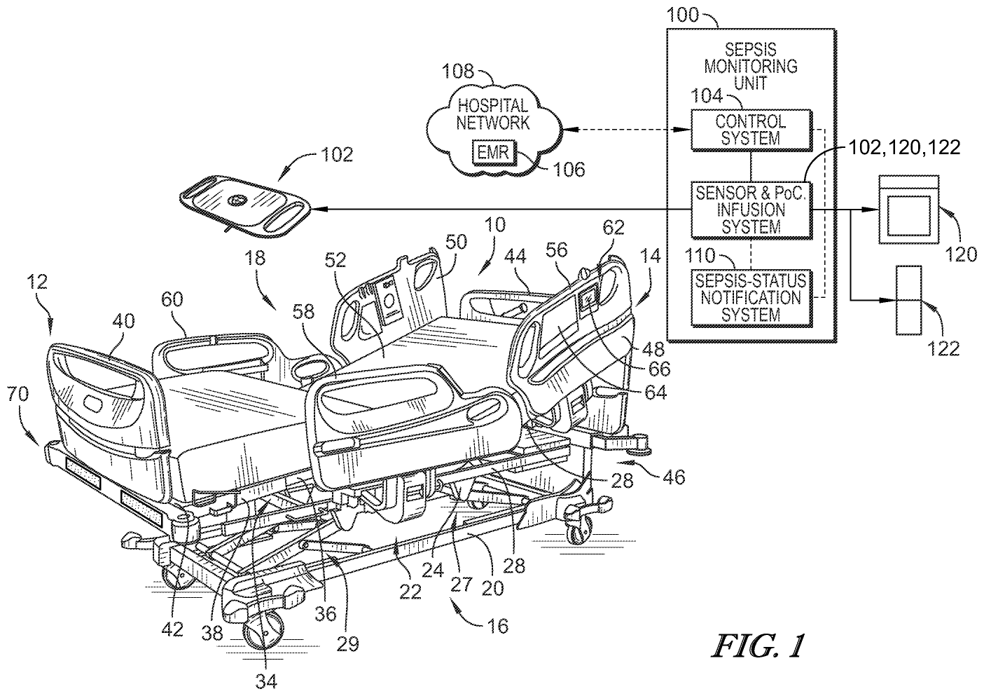

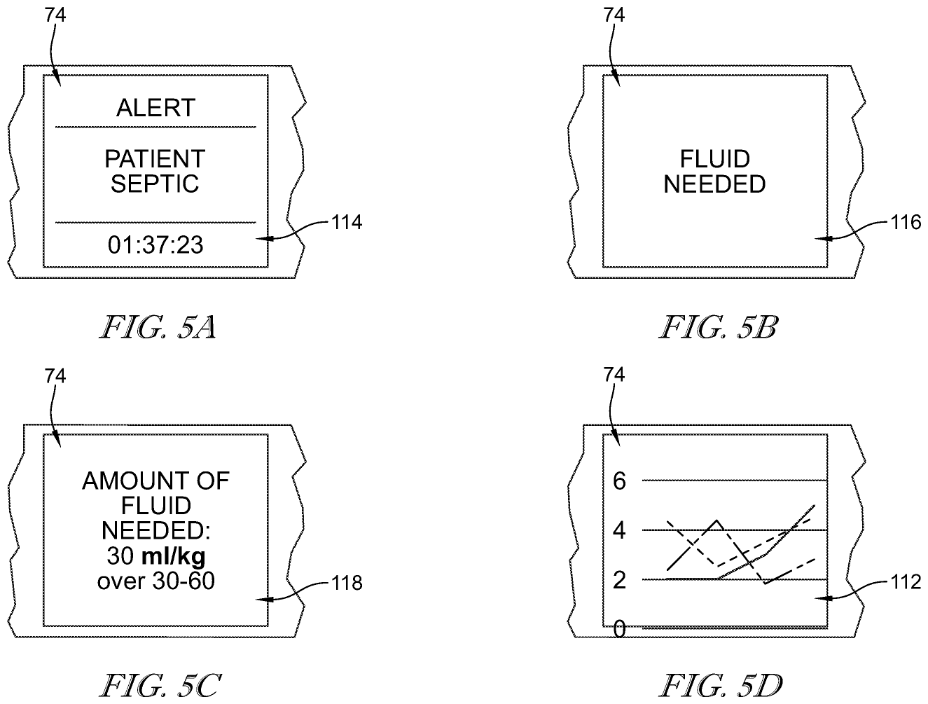

Referring to FIG. 1, a patient support apparatus 10 is illustratively embodied as a hospital bed 10. The hospital bed 10 includes an integrated sepsis monitoring unit 100 including a sepsis-status notification system 110. The sepsis monitoring unit 100 is configured to utilize data from a multitude of sources and, when appropriate, instruct the sepsis-status notification system 110 to provide an indication that the patient is at-risk for sepsis or has been diagnosed with sepsis 114 (as seen in FIG. 5A). If the patient is diagnosed with sepsis, the sepsis-status notification system 110 is also configured to monitor a status of a sepsis protocol administration and provide an indication of that status.

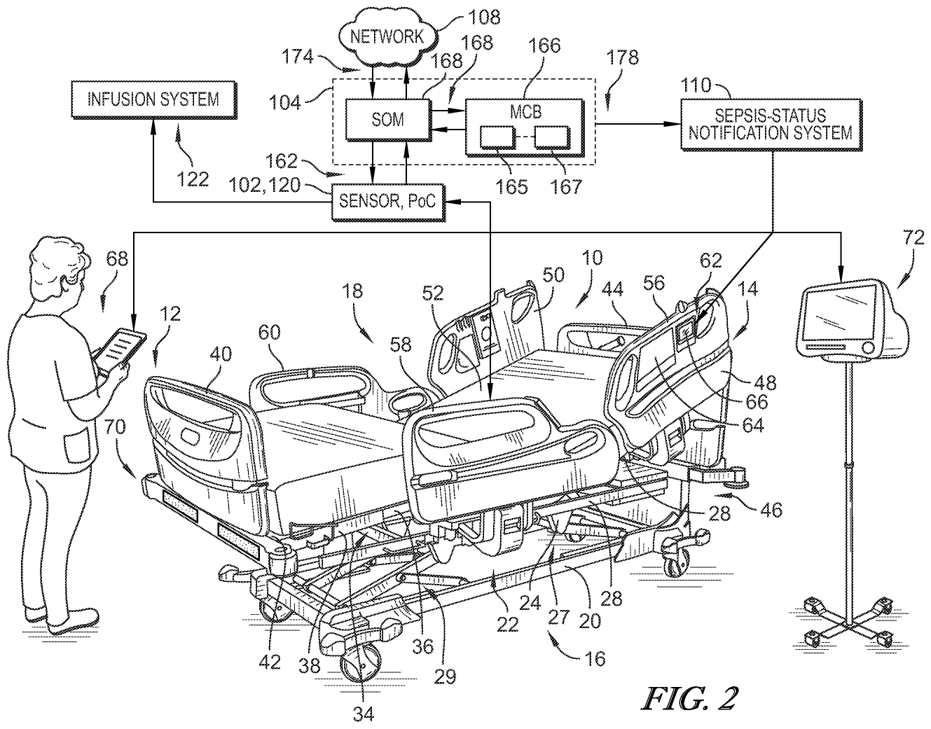

The view shown in FIGS. 1 and 2 is generally taken from a position that is oriented at the left side, foot end of the hospital bed 10. For purposes of orientation, the discussion of the hospital bed 10 will be based on the orientation of a patient supported on the hospital bed 10 in a supine position. Thus, the foot end 12 of the hospital bed 10 refers to the end nearest the patient's feet when the patient is supported on the hospital bed 10 in the supine position. The hospital bed 10 has a head end 14 opposite the foot end 12. A left side 16 refers to the patient's left when the patient is lying in the hospital bed 10 in a supine position. The right side 18 refers to the patient's right. When reference is made to the longitudinal length of the hospital bed 10, it refers a direction that is represented by the lines that generally extend between the head end 14 and foot end 12 of the hospital bed 10. Similarly, lateral width of the hospital bed 10 refers to a direction that is represented by the lines that generally extend between the left side 16 and right side 18.

The hospital bed 10 includes a base frame 20, which supports a lift system 22. The lift system 22 engages the base and an upper frame 24 such that the lift system 22 moves the upper frame 24 vertically relative to the base frame 20. The lift system 22 includes a head end linkage 27 and a foot end linkage 29. Each of the linkages 27 and 29 are independently is configured and can be operated to cause the hospital bed 10 to move into a tilt position which is when the head end 14 of the upper frame 24 is positioned lower than the foot end 12 of the upper frame 24. The hospital bed 10 can also be moved to a reverse tilt position with the foot end 12 of the upper frame 24 is positioned lower than the head end 14 of the upper frame 24.

The upper frame 24 supports a load frame 26. The load frame 26 supports a head deck 28, which is movable relative to the load frame 26. The load frame 26 also supports an articulated seat deck 30 (seen in FIG. 2), also movable relative to the load frame 26 and a fixed seat deck 32 (also seen in FIG. 2). Also supported from the load frame 26 is a foot deck 34 that is articulated and moveable relative to the load frame 26. The foot deck 34 in the illustrative embodiment of FIG. 1 provides for powered pivoting of the foot deck 34 and manual extension and retraction of the foot deck 34 to vary the length of the foot deck 34. In other embodiments, powered pivoting of the foot deck 34 can be omitted and the related movement can be caused manually, or follow movement of the articulated seat deck 30. In addition, in some embodiments, extension and retraction of the foot deck 34 can be powered by an actuator.

The foot deck 34 includes a first portion 36 and a second portion 38, which moves relative to the first portion 36 to vary the size of the foot deck 34. The second portion 38 moves generally longitudinally relative to the first portion 36 to vary the longitudinal length of the foot deck 34 and, thereby, the longitudinal length of the hospital bed 10.

A foot panel 40 is supported from the second portion 38 and extends vertically from an upper surface 42 of the second portion 38 to form a barrier at the foot end 12 of the hospital bed 10. A head panel 44 is positioned on an upright structure 46 of the base frame 20 and extends vertically to form a barrier at the head end 14 of the hospital bed 10. A left head side rail 48 is supported from the head deck 28 and is moveable between a raised position shown in FIG. 1 and a lowered position as is known in the art. A right head side rail 50 is also moveable between the raised position of FIG. 1 and lowered position. As shown in FIG. 1, in the raised position, the side rails 48 and 50 extend above an upper surface 52 of a mattress 54 of the hospital bed 10 when the side rails 48 and 50 are in a raised position. In a lowered position, an upper edge 56 of the left head side rail 48 is below the upper surface 52.

The hospital bed 10 also includes a left foot side rail 58 and a right foot side rail 60, each of which is supported directly from the load frame 26. Each of the side rails 48, 50, 58, and 60 are is configured to be lowered to a position below the upper surface 52. It should be noted that when the head deck 28 is moved, the head side rails 48 and 50 move with the head deck 28 so that they maintain their relative position to the patient. This is because both of the head side rails 48 and 50 are supported by the head deck 28.

In the illustrative embodiment, the hospital bed 10 further includes at least one sensor 102 mounted to a deck section of the hospital bed 10 and a control system 104 coupled to the sensor 102 as shown in FIG. 1. The sensor 102 is configured to provide sensor signals indicative of vital signs of the patient laying on the hospital bed 10. In one embodiment, the control system 104 is configured to receive the sensor signals and compare the sensor signals to pre-established acceptable conditions to determine if the patient is at-risk for developing a certain disease indication and/or is in compliance with a therapy.

In one embodiment, the control system 104 is configured to receive the sensor signals and compare the sensor signals to pre-established acceptable conditions to determine if the patient is at-risk or should be diagnosed with sepsis. In another embodiment, the sensor signals and information stored on the patient's EMR 106 are both compared to the pre-established acceptable conditions to determine if the patient is at-risk or should be diagnosed with sepsis.

In the illustrative embodiment, the sensor 102 is a non-contact vital signs monitoring sensor available from EarlySense Inc., 135 Beaver Street Suite 307, Waltham, MA 02452. It provides a signal indicative of vital signs, such as, for example, a detected heart rate and a signal indicative of a detected respiration rate that is processed by the control system 104. This sensor 102 is described in greater detail in U.S. Patent Pub. No. 2018/0184984, which is expressly incorporated herein for the purpose of describing a suitable sensor used to detect patient vital signs. The sensor 102 can be mounted in multiple locations on either the fixed seat deck 32 or head deck 28 as suggested in FIG. 1. In some embodiments, other vital sign monitoring sensors can be used with the patient support apparatus 10. The sensor 102 can monitor blood pressure, heart rate, respiratory rate, and/or blood oxygen saturation level.

In some embodiments, multiple sensors 102 can be positioned on the fixed seat deck 32 and/or head deck 28 to provide multiple detection points with the signals from each of the multiple sensors 102 being monitored to determine an accurate vital sign signal. The use of redundant signals reduces the risk of signal loss due to movement or improper positioning of the patient on the hospital bed 10. The sensor 102 has a relatively thin thickness that permits the sensor 102 to be placed under the mattress 54 and does not interfere with the functionality or therapeutic benefit of the mattress 54. In other embodiments, a different piezoelectric sensor can be utilized in place of the sensor 102. In another embodiment, the sensor 102 can be positioned inside of the mattress 54. In other embodiments, additional sensors can be wearable by the patient and communicatively connected to the control system 104 to provide signals indicative of vital signs of the patient.

In one embodiment, the hospital bed 10 further includes a point of care (PoC) device 120 to collect date to determine the occurrence of a disease indication or compliance with a therapy in conjunction with the vital sign(s) determined by the sensor 102. In the illustrative embodiment, the hospital bed 10 the point of care (PoC) device 120 can be utilized to collect date to determine the occurrence of sepsis in conjunction with the vital sign(s) determined by the sensor 102. The point of care (PoC) device 120 can be a peripheral device, can be attached to the bed 10, can be integrated with the hospital bed 10, can be connected directly to the sepsis monitoring unit 100, and/or can be movable by a caregiver. The point of care (PoC) device 120 can be a blood cell type or blood cell quality counter (e.g., a white blood cell counter), or a cardiac monitor that can acquire and analyze the patient's ECG. The point of care (PoC) device 120 can be an independent device including but not limited to a WBC diagnostic tool (e.g., Athelas etc.), a cardiac monitor (e.g., Cheetah medical, Baster Mortara, Baxter ToSense etc.). The point of care (PoC) device 120 can integrate a blood test module to detect blood cell counts. In some embodiments, the point of care (PoC) device 120 can acquire white blood cell count of the patient in less than about a minute. The point of care (PoC) device 120 can include disposable tools (e.g., finger sticks, glass slides) to assess a drop of blood from the patient. The point of care (PoC) device 120 can include module trays for assessing patient blood, and a location for storing the disposable tools.

Sepsis can be predicted or detected using risk assessments that can vary between each healthcare facility. Some non-limiting examples of scores used to determine if a patient is at-risk for sepsis include a quick sequential (sepsis-related) organ failure assessment score (qSOFA) and/or systematic inflammatory response syndrome score (SIRS). Each method includes a set of criteria such as vital signs and/or other conditions that are defined by pre-established acceptable limits and are triggered when the patient deviates from those acceptable limits. These conditions can be monitored by the point of care (PoC) devices 120. Other examples of assessments or tests used to determine if a patient is at risk for sepsis are described in U.S. Provisional Patent Appl. No. 62/655,385, filed Apr. 10, 2018, which is expressly incorporated by reference herein. The sepsis-status notification system 110 can be programmed to operate with any assessment, test, or score criteria relating to sepsis including those that evolve as medical procedures advance. In some embodiments, the wearable device or sensor can be specifically designed to detect the presence of sepsis such that the scoring methods described above are omitted and/or used in conjunction with the wearable device.

The sepsis protocol can include a therapy operation protocol. The sepsis protocol can include a list of actions or procedures that should be administered to the patient within a certain time period (e.g., within a three hour period and/or a six hour period beginning at the time the patient is diagnosed with sepsis or otherwise determined to be septic). For example, the sepsis protocol may include that within the three-hour protocols and timeframe, the following actions should be taken: 1) obtain blood cultures, 2) obtain a lactate measurement, 3) administer broad spectrum antibiotics, and 4) administer fluids crystalloid. Within a six-hour protocol and timeframe, the following additional actions should be taken: 1) administration of vasopressors for non-responsive resuscitation, 2) maintaining adequate central venous pressure (CVP) and mixed venous oxygen content (CvO2) levels, and 3) obtaining a second lactate measurement if the first lactate measurement was high. Other sepsis risk assessment and sepsis treatment protocols can be used. For example, a sepsis protocol may include fluid administration to the patient.

Compliance with the sepsis protocols can increase the patient's chance of survival after being diagnosed with sepsis. The sepsis-status notification system 110 is configured to increase compliance with the protocols by notifying the caregiver and others near the patient if the patient is at-risk or diagnosed with sepsis, an amount of time that has passed, and a status of the actions that need to be completed for compliance with the protocols.

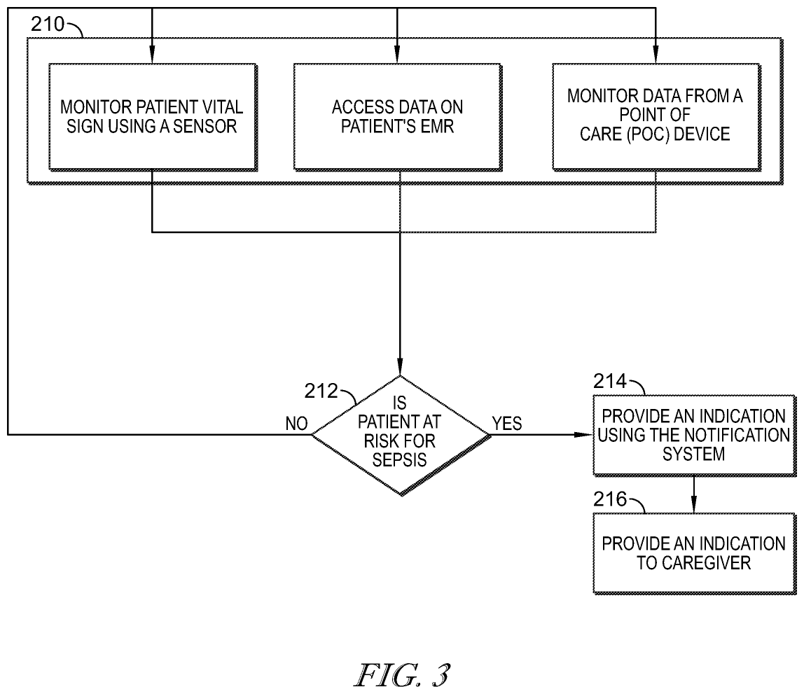

As shown in FIG. 3, the sepsis monitoring unit 100 uses the sensor 102, the point of care (PoC) device 120, and the control system 104 to monitor the patient's risk for sepsis at step 210. At step 212, the sepsis monitoring unit 100 then determines whether the patient is at-risk for sepsis or should be diagnosed with sepsis based on the vital sign signals provided by the sensor 102, the point of care (PoC) device 120, and/or the information stored on the patient's EMR 106. The vital signs and the information on EMR 106 are compared to the pre-established acceptable limits to assess or predict if the patient is at risk for sepsis. At step 214, the control system 104 can send a command signal as soon as the patient is determined to be at-risk for sepsis to cause the sepsis-status notification system 110 to indicate to a caregiver that the patient is septic and start monitoring the protocols. The indication provided by the sepsis-status notification system 110 can include a visual indication or an audible indication. Additionally, the indication can be displayed on or around the hospital bed 10 or transmitted wirelessly to a remote location through the hospital network 108.

Once the patient is determined to be at-risk or diagnosed with sepsis, the control system 104 sends a command signal to the sepsis-status notification system 110 to cause the sepsis-status notification system 110 to provide the indication to the caregiver at step 216. The sepsis monitoring unit 100 continues monitoring and updating the indications provided by the sepsis-status notification system 110 in real time until administration of the protocols is completed.

Fluid administration for sepsis patients is critical and can be accomplished non-invasively. For example, a Baxter Starling Fluid Management System can be employed provide recommendation for fluid administration to sepsis patients. Such a system combines cardiac monitoring with patient positioning by measuring bio-impedance and calculating a full hemodynamic profile including stroke volume and cardiac output when determining if fluid administration is needed (as seen in FIG. 5B). In one embodiment, the accuracy of fluid management can be enhanced by combing vital signs monitoring by the sensor 102 with measurements made by a cardiac monitor, which is a point of care (PoC) device 120. Alternatively, or additionally, artificial intelligence (AI) or data driven clinical decision support can be used in addition to the cardiac monitor when recommending a precise amount of intravenous (IV) fluid for the sepsis patient. This could lead to faster recovery from sepsis while mitigating adverse events due to over administration of IV fluid.

In one embodiment, the control system 104 calculates and displays sepsis risk severity and an optimal fluid challenge amount for the patient using a combination of vital signs being monitored by the sensor 102 and the point of care (PoC) device 120 (e.g., cardiac monitor) (as seen in FIG. 5C). The hospital bed 10 can include a Baxter Starling cardiac monitor, a Baxter ToSense cardiac monitor, a Baxter Mortara cardiac monitor, or any other cardiac monitor compatible with the control system 104. The point of care (PoC) device 120 (e.g., cardiac monitor) can communicate with a patient infusion system 122 (e.g., Baxter Spectrum IQ Infusion System) to automatically adjust fluid input to the patient.

The control system 104 can determine an accurate estimate of fluid that needs to be administered based on data obtained from the sensor 102 and the point of care (PoC) device 120 (e.g., cardiac monitor). The sepsis-status notification system 110 can display a visual or audible indication that the patient is septic by using a plurality of devices. Additionally, or alternatively, the sepsis-status notification system 110 can display a visual or audible indication about fluid administration and/or fluid responsiveness of the patient. For example, the sepsis-status notification system 110 can display a sepsis graph indicating a rate of improvement in the patient and indicate fluid responsiveness of the patient (as seen in FIG. 5D). In some embodiments, such indication can be directly displayed on the sensor 102, the point of care (PoC) device 120, the patient infusion system 122, or a separate user interface 62.

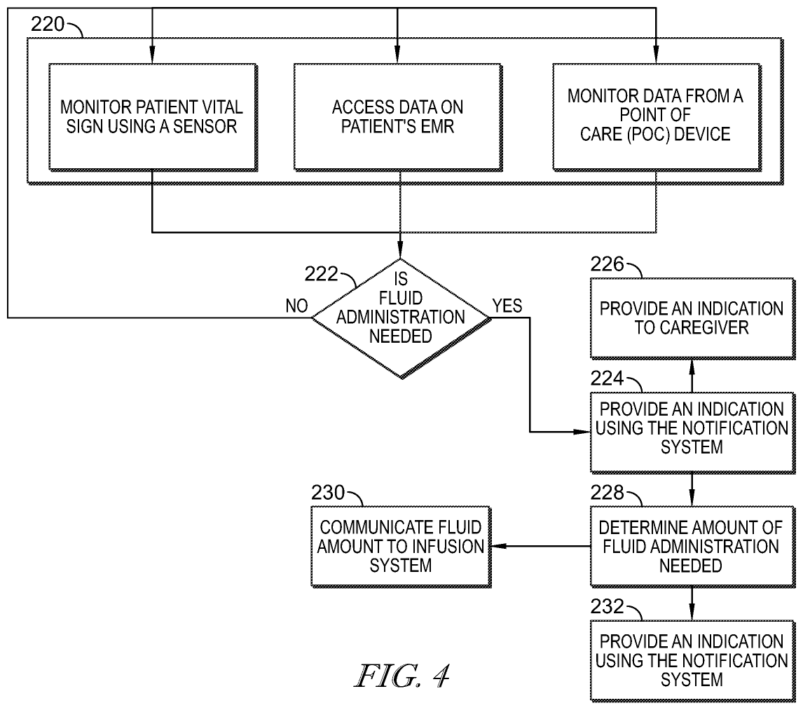

As shown in FIG. 4, the sepsis monitoring unit 100 uses the sensor 102, the point of care (PoC) device 120, and the control system 104 to accurately estimate fluid administration to the patient at step 220. At step 222, the sepsis monitoring unit 100 then determines if fluid administration is needed based on the vital sign signals provided by the sensor 102, the point of care (PoC) device 120, and/or the information stored on the patient's EMR 106. At step 224, the control system 104 can send a command signal as soon as the patient is determined to need fluid administration to the notification system 110 to indicate to fluid administration is needed. The indication provided by the sepsis-status notification system 110 can include a visual indication or an audible indication.

The control system 104 sends a command signal to the sepsis-status notification system 110 to cause the sepsis-status notification system 110 to provide the indication to the caregiver at step 226. Additionally, the indication can be displayed on or around the hospital bed 10 or transmitted wirelessly to a remote location through the hospital network 108. The control system 104 can determine the amount of fluid required for administration in step 228, and can communicate that amount to the infusion system 122 in step 230. The control system 104 sends a command signal to the sepsis-status notification system 110 to cause the sepsis-status notification system 110 to provide the indication to the caregiver about the amount of fluid required for administration at step 232. The sepsis monitoring unit 100 continues monitoring and updating the indications provided by the sepsis-status notification system 110 in real time until administration of the protocols is completed.

As shown in FIG. 2, the control system 104 provides all of the functionality necessary to operate the sepsis monitoring unit 100 and includes a system on a module (SOM) 164 and a master controller (MCB) 166. The sensor 102 and the point of care (PoC) device 120 communicate through a universal asynchronous receiver/transmitter (UART) connection 162 with the SOM 164. The SOM 164 is connected to and communicates with the MCB 166 through a UART connection 168. The SOM 164 is configured to communicate with hospital network 108 through a connection 174 and can connect to external systems, such as nurse call systems or other hospital wide communications systems such as the NaviCare® system from Hill-Rom Company, Inc., Batesville, IN, using the hospital network 108. The sensor 102, the point of care (PoC) device 120, and the hospital network 108 can communicate through manual entry, wired, wireless and/or power line connections and associated protocols (e.g., Ethernet, InfiniBand®, Bluetooth®, Wi-Fi®, WiMAX, 3G, 4G LTE, 5G, etc.). This allows information regarding the vital signs detected, including alarm conditions, and/or information collected from the point of care (PoC) device 120 to be transferred to other locations in the hospital or other facility in which the hospital bed 10 is located.

In one embodiment, the MCB 166 communicates with each device through a connection 178 or wirelessly using the SOM 164 and the hospital network 108. The connection 178 can be a simple UART interface, a CAN interface, a discrete wiring connection, or any other suitable connection. Each of the connections 162, 168, 170, 174, 178, and 184 can be a simple UART interface, a CAN interface, a discrete wiring connection, or any other suitable connection as required for the particular application. Relative to the sepsis-status notification system 110, the MCB 166 includes a processor 165 and a non-transitory memory device 167 that stores instructions. When appropriate, the instructions are executed to operate the sepsis monitoring unit 100 and display the indications. Some of the processing and instructions can be resident on the SOM 164 as it relates to specific tasks to be executed under the direction of the MCB 166.

Referring to the left head side rail 48 shown in FIG. 2, the user interface 62 includes a hard panel 64 and a graphical user interface 66. The hard panel 64 provides indications to a user regarding the status of certain functions of the hospital bed 10 as well as providing a standard set of fixed input devices. The graphical user interface 66 includes a touchscreen display that provides information to a user as well as allowing for flexible, menu driven, operation of certain functions of the hospital bed 10. The graphical user interface 66, also known as a flip-up display (FUD), is mounted to the side rail 48 with a pivotable connection so that the graphical user interface 66 can be pivoted to allow a user the more easily view and interact with the graphical user interface 66. In some embodiments, the right head side rail 50 can include a second graphical user interface duplicative of the graphical user interface 66.

As shown in FIG. 2, the sepsis-status notification system 110 can display the indications using the graphical user interface 66, a mobile device 68 used by the caregiver, an indicator panel 70 on the hospital bed 10, and/or one or more monitors 72 located in the room where the hospital bed 10 and the patient are located. The mobile device 68 is illustratively embodied as a tablet, such as an iPad®, that is carried by the caregiver during rounds. The indicator panel 70 is coupled to the foot end 12 of the hospital bed 10, but can be located on any part of the hospital bed 10 that is easily viewable. The monitor 72 is illustratively embodied as a vitals monitor but can also include a computer monitor or a television screen located in the room. Other devices located in a remote location, such as, for example, a nurse call station, can also be used. As shown in FIG. 5A-5D, the indicator panel 70 is configured to display several visual indications that indicate that the patient is septic and if action is needed. As shown in FIG. 5A, indicator panel 70 can include a first section 76 with a light that is displayed in a first color prior to the patient being diagnosed with sepsis.

The sepsis-status notification system 110 can use the other devices 66, 68, and 72 to indicate that the patient is septic and action is needed. The sepsis-status notification system 110 can use the other devices 66, 68, and 72 to indicate that the patient needs fluid administration and/or the amount of fluid that needs to be administered. The MCB 166 is configured to send a command signal to one or more of the devices 66, 68, 72 to cause the devices to display an alert message 74 when the patient is diagnosed with sepsis. The MCB 166 can also send a command signal to one or more of the devices 66, 68, 72 to cause the devices to provide an audible alert to notify the caregiver to check the device or to audibly convey the alert message 74. As shown in FIGS. 5A-5D, the alert message 74 includes information indicating to the caregiver that the patient is septic 114 and information related to the status of the sepsis protocols administration. For example, each time the patient is scanned during rounds or connected to a new device with a monitor, the MCB 166 is configured to send the command signal to the device to cause the alert message 74 to be displayed. Alternatively, the alert message 74 includes information indicating to the caregiver that the patient needs fluid administration 116, the amount of fluid required for administration 118, or patient rate of improvement 112. Additionally, the alert message 74 can be displayed multiple times during protocol administration to remind caregivers of actions to be taken as described above.

Although this disclosure refers to specific embodiments, it will be understood by those skilled in the art that various changes in form and detail can be made without departing from the subject matter set forth in the accompanying claims.

1. A patient support apparatus comprising:
a sensor providing a first signal indicative of a vital sign of a patient positioned on the patient support apparatus;
a point of care device providing a second signal; and
a controller coupled to the sensor and the point of care device, including a processor and a memory device, the memory device including instructions that, when executed, cause the controller to receive the first signal from the sensor and the second signal from the point of care device, analyze the first and the second signals to determine data indicative of sepsis, compare the data indicative of sepsis to pre-established acceptable limits, and if the patient is assessed to have a risk for sepsis or has been diagnosed with sepsis, output a command signal.

2. The patient support apparatus of claim 1, further comprising a sepsis notification system responding to the command signal from the controller that displays a first visual indication showing if the patient has risk for sepsis or has been diagnosed with sepsis.

3. The patient support apparatus of claim 1, wherein the point of care device is a peripheral device or a connected device.

4. The patient support apparatus of claim 1, wherein the point of care device is a cell counter, a cell quality monitor, or a cardiac monitor.

5. The patient support apparatus of claim 1, wherein the point of care device is a blood test module and comprises disposable tools for testing blood cell count or blood cell quality.

6. The patient support apparatus of claim 1, wherein the controller is configured to communicate with an external nurse call station.

7. The patient support apparatus of claim 1, wherein the controller is configured to communicate with an electronic medical record system to receive information from the electronic medical record system indicative of a medical history of a patient supported on the patient support apparatus.

8. A patient support apparatus comprising:
a sensor providing a first signal indicative of a first vital sign of a patient supported on the patient support apparatus;
a point of care device providing a second signal indicative of a cardiac output of the patient; and
a controller coupled to the sensor and the point of care device, and including a processor and a memory device, the memory device including instructions that, when executed, cause the controller to receive the first and second signals, calculate a sepsis risk severity and an optimal fluid challenge amount based on the first and second signals.

9. The patient support apparatus of claim 8, wherein the controller further receives and analyzes information from an electronic medical record storing patient information for the patient.

10. The patient support apparatus of claim 8, wherein the apparatus further comprises a notification system responding to the command signal from the controller to provide a first visual indication that shows that the patient has risk for sepsis or has been diagnosed with sepsis.

11. The patient support apparatus of claim 8, wherein the apparatus further comprises a notification system responding to the command signal from the controller to provide a first indication that shows that the patient has risk for sepsis or has been diagnosed with sepsis, and a second indication that shows an amount of fluid needed for fluid administration.

12. The patient support apparatus of claim 11, wherein the apparatus further comprises the controller communicating with a patient infusion system to automatically adjust fluid input to the patient.

13. The patient support apparatus of claim 11, wherein the notification system provides a third indication that shows a sepsis graph indicating a rate of improvement in the patient.

14. A method of predicting sepsis in a patient on a patient support apparatus comprising an inflatable mattress, the method comprising the steps of:
monitoring a first signal from a sensor, wherein the first signal is indicative of a vital sign of the patient;
monitoring a second signal from a point of care device;
processing the first signal and the second signal to determine data indicative of sepsis;
comparing the data indicative of sepsis to pre-established acceptable limits to assess if the patient has sepsis or has a risk for sepsis; and
outputting a command signal to indicate if the patient has sepsis or has a risk for sepsis.

15. The method of claim 14, wherein processing the first signal and the second signal to determine data indicative of sepsis comprises a controller analyzing the first and the second signals to determine data indicative of sepsis and comparing the data indicative of sepsis to pre-established acceptable limits.

16. The method of claim 15, wherein processing the first signal and the second signal further comprises the controller receiving and analyzing information from an electronic medical record storing patient information for the patient.

17. The method of claim 15, wherein the method further comprises a notification system responding to the command signal from the controller to provide a first visual indication that shows that the patient has sepsis or has a risk for sepsis.

18. The method of claim 17, wherein the method further comprises the notification system responding to the command signal from the controller to provide a second visual indication that shows if the patient needs fluid administration.

19. The method of claim 18, wherein the method further comprises the notification system responding to the command signal from the controller to provide a third visual indication that shows an amount of fluid needed for fluid administration.

20. The method of claim 19, wherein the method further comprises the controller communicating with a patient infusion system to automatically adjust fluid input to the patient.
